(19) United States
(12) Patent Application Publication
(10) Pub. No.: US 2006/0020234 A1
(43) Pub. Date: Jan. 26, 2006

(54) Absorbent sac wound dressing

(76) Inventors: Lin-Shing Chou, Taipei (TW); Wen-Hsiang Huang, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/895,358

(22) Filed: Jul. 21, 2004

(51) Int. Cl. A61F 13/00

(52) U.S. Cl. 602/41; 602/43; 602/47

(57) ABSTRACT

An absorbent sac wound dressing comprising a wound-contacting layer covered with tapered pores and the bottom surface of tapered pores contacting a wound area wherein discharged exudate penetrates through, a guiding layer transmitting discharged exudate to an absorbent layer, an absorbent layer absorbing discharged exudate to make fibers expand into the shape of gel, which is effective in preventing from backflow of exudate to a wound area, and a translucent breathing layer having a broad spread of micro pores. The placement of the above layer is one on top of another in order and the peripheral edges are joined together by heat-sealing to form a sac without side escape. More particularly, the certain concentration of water-soluble antimicrobial medicines, enzymes or growth factor agents in a suitable amount are well distributed added in the absorbent layer, which is more effective in controlling a wound infection.

### BACKGROUND OF THE INVENTION

1. Field of the Invention

The present invention relates to an absorbent sac wound dressing and more particularly to a wound-contacting layer of the absorbent sac wound dressing, which has a porous film covered with tapered pores wherein wound exudate can penetrate through to prevent from backflow of exudate without reinjury to a wound. Also, the certain concentration of water-soluble antimicrobial medicines, enzymes or growth factor agents in a suitable amount are well distributed added in an absorbent layer of the absorbent sac wound dressing, which can be more effective in controlling a wound infection, debridment and promoting a wound healing.

2. Prior Art

Almost every healing process generally used in the medical institution is necessarily using antimicrobial dressings to cover or bandage a wound for preventing from infection with the external bacteria and the wound is possible to be the laceration, surgical incision, inflammation or burns. In addition, most commonly used dressings in the medical institution are still gauze or cotton wad that is made of antimicrobial cotton positioned between two layers of gauze. However, these dressings may be effective in absorbing wound exudate while directly contacting the wound but wound exudate may flow backward to infect the wound, or the absorbed exudate may evaporate easily so that gauze becomes harder to hurt the wound. Moreover, if gauze is adhering to the wound while removing the dressings, a healing process may be hindered by causing a reinjury. Accordingly, it is beneficial to provide the absorbent sac wound dressing of the present invention.

### OBJECTS OF THE INVENTION

The objects of the present invention are to solve said disadvantages and more specifically to provide an absorbent sac wound dressing. According to the present invention, the absorbent sac wound dressing comprising:
(a) a wound-contacting layer having a porous film covered with tapered pores, the bottom surface of tapered pores with the largest caliber contacting the wound area wherein exudate from a wound rapidly penetrates through in a one-way direction to prevent from backflow of exudate.
(b) a guiding layer sealed on said wound-contacting layer having directional fibers or non-woven fabrics to transmit said wound exudate to the absorbent layer;
(c) an absorbent layer sealed on said guiding layer having a high-molecule polymeric absorbent material;
(d) a breathing layer sealed on said absorbent layer having a translucent waterproof film to protect the wound from infection, wherein people can observe the absorbing condition of the dressing.

In the preferred embodiment, the certain concentration of water-soluble antimicrobial medicines, enzymes or growth factor agents in a suitable amount are well distributed added in the absorbent layer.

As the absorbent sac wound dressing of the present invention is applied on a cavity-type wound or an infectious wound, it is effective in controlling a wound infection by backflow of the certain concentration of agents even if the dressing has been in a saturated state.

As the absorbent sac wound dressing of the present invention is applied on a wound having necrotic tissue, it is effective in cleansing the wound by backflow of enzymes to disintegrate necrotic tissue even if the dressing has been in a saturated state.

As most generally used dressings are applied on an infectious wound or a wound having necrotic tissue, dressings have to be removed frequently or the wound may get more serious. Therefore, the absorbent sac wound dressing of the present invention is to use backflow of the certain concentration of agents to the wound area to complete the effect of inhibiting bacteria growth, disintegrating necrotic tissue and even promoting a newborn granulation tissue growth.

It is obviously known that the absorbent sac wound dressing of the present invention can complete the effect of controlling infection, debridment or promoting a wound healing; also, the frequency of changing the dressing can be reduced.

The present invention can be formed into various shapes and its shape can be applied on different kinds of the wound.

### BRIEF DESCRIPTION OF THE DRAWINGS

These and other features, aspects and advantages of the present invention will become apparent from the following description, claims and the accompanying embodiments shown in the drawings, which are briefly described below.

FIG. 1 is an exploded perspective view of the absorbent sac wound dressing of the present invention.

FIG. 2 is an enlarged cross-sectional view of the absorbent sac wound dressing of the present invention.

### DESCRIPTION OF THE PREFERRED EMBODIMENT

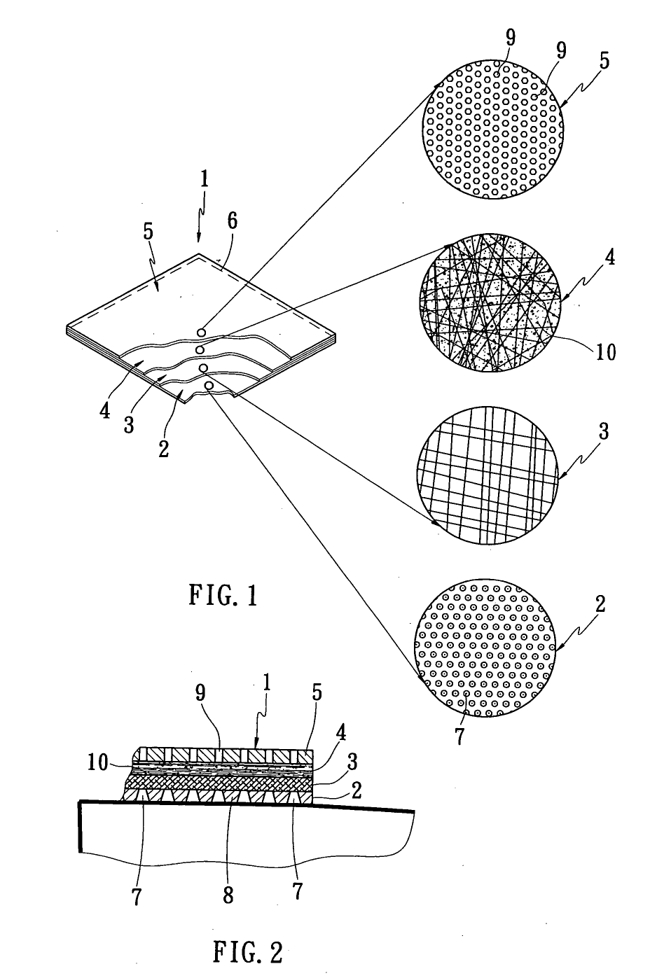

Referring to FIG. 1 and FIG. 2, the absorbent sac wound dressing (1) of the present invention comprises a wound-contacting layer (2), a guiding layer (3), an absorbent layer (4) and a breathing layer (5). The placement of the above layer is one on top of another in order and the peripheral edges are joined together by heat-sealing (6). The absorbent sac wound dressing has various sizes in accordance with the different size of a wound. Wherein the wound-contacting layer (2) has a porous film covered with tapered pores (7). Said film can be a non-adhering polyethylene film or other similar materials and said tapered pores (7) can be polygonal tapered pores or conical shaped pores. The bottom surface of tapered pores (7) with the largest caliber contacts a wound area (8) (as shown in FIG. 2) to produce a one-way valve and discharged exudate from the wound area (8) is able to penetrate through numerous tapered pores (7). And said discharged exudate is transmitted via the guiding layer (3) to the absorbent layer (4) forming into the shape of gel to prevent from backflow. The certain concentration of antimicrobial medicals, enzymes or growth factor agents in a suitable amount are added in a dry absorbent material of the absorbent layer (4) and said certain concentration is considered by the saturation capacity of said absorbent material. After the absorbent material absorbs discharged exudate being in a saturated state, the certain concentration of dissolved antimicrobial medicines, enzymes or agents will flow backward to the wound area (8).

As the absorbent sac wound dressing is applied on a cavity-type wound or infectious wound, it is effective in controlling a wound infection; as the absorbent sac wound dressing is applied on a wound having necrotic tissue, backflow of dissolved enzymes in the absorbent layer (4) can disintegrate necrotic tissue to complete the effect of debridment and also growth factor agents can promote a wound healing.

In the present embodiment, the wound-contacting layer (2) is made from a soft film covered with tapered pores. Besides having the advantage of producing a one-way valve, it is also not adhering to the wound; for this reason, the dressing is removed easily during a healing process to avoid hurting the newborn epidermis.

As shown in FIG. 1 and FIG. 2, the guiding layer (3), joined to the upper of the wound-contacting layer (2), can be directional non-woven fabrics to transmit said discharged exudate to the absorbent layer (4). Said absorbent layer (4) has the absorbent material that can be high-molecule polymer, such as seaweed gel. After absorbing discharged exudation, high-molecule polymeric fibers expand to form in the shape of gel. The absorbent material is added with the certain concentration of said agents in a suitable amount to absorb discharged exudate. After being in a saturated state, the certain concentration of dissolved agents will flow backward to the wound area (8). In the present embodiment, the absorbent material is made from non-woven pulp and high-molecule polymeric fiber under an antiseptic treatment. The breathing layer (5), above the absorbent layer (4), having a translucent film with a broad spread of micro pores (9) to keep the wound in suitable moisture and people can observe the absorbing condition of the dressing (1) through said breathing layer (5).

1. An absorbent sac wound dressing, comprising:
a) a wound-contacting layer having a suitable soft film covered with tapered pores, the bottom surface of tapered pores with the largest caliber contacting a wound area wherein discharged exudate penetrates through to prevent from backflow of exudate to the wound;
b) a guiding layer sealed on said wound-contacting layer having directional fibers or non-woven fabrics to transmit said discharged exudate to an absorbent layer;
c) an absorbent layer sealed on said guiding layer having a high-molecule polymeric absorbent material to absorb said discharged exudation; and
d) a breathing layer sealed on said absorbent layer having a translucent film with a broad spread of micro pores for air venting to keep the wound in suitable moisture; also people can observe the absorbing condition of the dressing through said breathing layer.

2. An absorbent sac wound dressing as claimed in claim 1 wherein said absorbent material in said absorbent layer is added with the certain concentration of water-soluble antimicrobial medicines, enzymes or growth factor agents in a suitable amount and said certain concentration is considered by the saturation capacity of said absorbent material. After said absorbent material absorbs discharged exudate being in a saturated state, the certain concentration of dissolved antimicrobial agents will flow backward to a wound area to complete the effect of controlling a wound infection, debridment and promoting a wound healing.
